(19) United States
(12) Patent Application Publication
(10) Pub. No.: US 2021/0052211 A1
(43) Pub. Date: Feb. 25, 2021

(54) APPARATUS FOR IMAGING THE PROSTATE

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 17/081,475

(22) Filed: Oct. 27, 2020

Related U.S. Application Data
(63) Continuation of application No. PCT/CN2018/087624, filed on May 21, 2018.

(51) Int. Cl. A61B 5/00

(52) U.S. Cl. CPC A61B 5/4381; A61B 5/0084; A61B 5/0062; A61B 5/6847

(57) ABSTRACT

Disclosed herein is an apparatus comprising: an insertion tube configured to be inserted into a human; a metal target disposed inside the insertion tube and configured to emit X-ray by receiving radiation.

### BACKGROUND

The prostate is a gland of the male reproductive system in human. The prostate secretes a slightly alkaline fluid that constitutes about 30% of the volume of semen. The alkalinity of semen helps prolonging the lifespan of sperms. Prostate diseases are common, and the risk increases with age. Medical imaging (e.g., radiography) can help diagnosis of prostate diseases. However, because the prostate is deep inside the human body, imaging the prostate may be difficult. For example, the thick tissues around the prostate may reduce the imaging resolution or increase the dose of radiation sufficient for imaging.

### SUMMARY

Disclosed herein is an apparatus comprising: an insertion tube configured to be inserted into a human; a metal target disposed inside the insertion tube and configured to emit X-ray by receiving radiation.

According to an embodiment, the insertion tube is configured to be inserted into the rectum of the human.

According to an embodiment, the metal target is configured to be inside the human when the insertion tube is inserted into the human.

According to an embodiment, the metal target is configured to move along the insertion tube.

According to an embodiment, the metal target is configured to rotate relative to the insertion tube.

According to an embodiment, the metal target comprises an angled surface configured to receive the radiation.

According to an embodiment, the radiation is electrons.

According to an embodiment, the apparatus further comprises an electron emitter disposed inside the insertion tube, configured to emit the electrons, and configured to accelerate the electrons toward the metal target.

According to an embodiment, the electron emitter is configured to be left outside of the human when the insertion tube is inserted into the human.

According to an embodiment, the radiation is an electromagnetic radiation.

According to an embodiment, the electromagnetic radiation is another X-ray.

According to an embodiment, the metal target is configured to emit X-ray due to fluorescence caused by the electromagnetic radiation.

According to an embodiment, the apparatus further comprises polycapillary lenses configured to direct the electromagnetic radiation toward the metal target.

According to an embodiment, the apparatus further comprises a radiation source configured to produce the electromagnetic radiation.

According to an embodiment, the radiation source is configured to be left outside of the human when the insertion tube is inserted into the human.

According to an embodiment, photons of the X-ray have energies between 20 keV and 30 keV.

According to an embodiment, the apparatus further comprises an image sensor configured to capture an image of a portion of the human using the X-ray.

Disclosed is a method comprising: inserting an insertion tube with a metal target therein into a human; emitting X-ray from the metal target by directing radiation onto the metal target; imaging a portion of the human with the X-ray.

According to an embodiment, inserting the insertion tube into the human comprises inserting the insertion tube into the rectum of the human.

According to an embodiment, the portion is the prostate.

According to an embodiment, the method further comprises moving the metal target along the insertion tube or rotating the metal target with respect to the insertion tube.

According to an embodiment, photons of the X-ray have energies between 20 keV and 30 keV.

According to an embodiment, the radiation is electrons.

According to an embodiment, the method further comprises producing the electrons outside the human.

According to an embodiment, the radiation is an electromagnetic radiation.

According to an embodiment, the electromagnetic radiation is another X-ray.

According to an embodiment, emitting X-ray from the metal target is by fluorescence of the metal target caused by the electromagnetic radiation.

According to an embodiment, the method further comprises producing the electromagnetic radiation outside the human.

According to an embodiment, directing the radiation onto the metal target is by using polycapillary lenses.

### BRIEF DESCRIPTION OF FIGURES

FIG. 1A and FIG. 1B each schematically show a functional diagram of an apparatus, according to an embodiment.

FIG. 2A and FIG. 2B each schematically shows a system including the apparatus, according to an embodiment.

FIG. 3 schematically shows that an image sensor having an array of pixels, according to an embodiment.

FIG. 4A shows a cross-sectional schematic of the image sensor, according to an embodiment.

FIG. 4B shows a detailed cross-sectional schematic of the image sensor, according to an embodiment.

FIG. 4C shows an alternative detailed cross-sectional schematic of the image sensor, according to an embodiment.

FIG. 5A and FIG. 5B each show a component diagram of an electronic system of the image sensor, according to an embodiment.

FIG. 6 schematically shows a temporal change of the electric current flowing through an electric contact (upper curve) of the radiation absorption layer of the image sensor, and a corresponding temporal change of the voltage on the electric contact (lower curve).

FIG. 7 shows a flowchart for a method using the apparatus, according to an embodiment.

### DETAILED DESCRIPTION

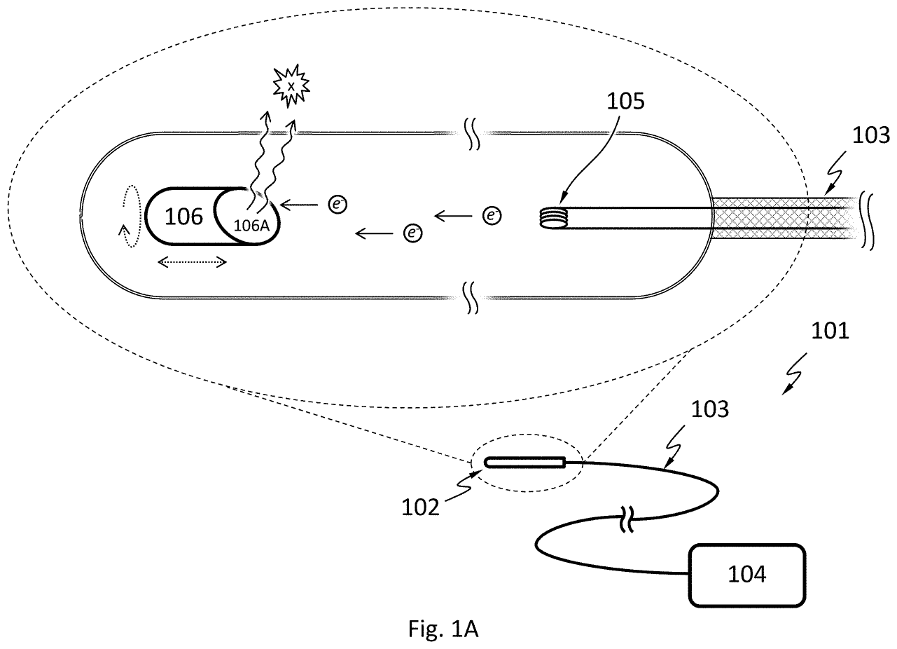

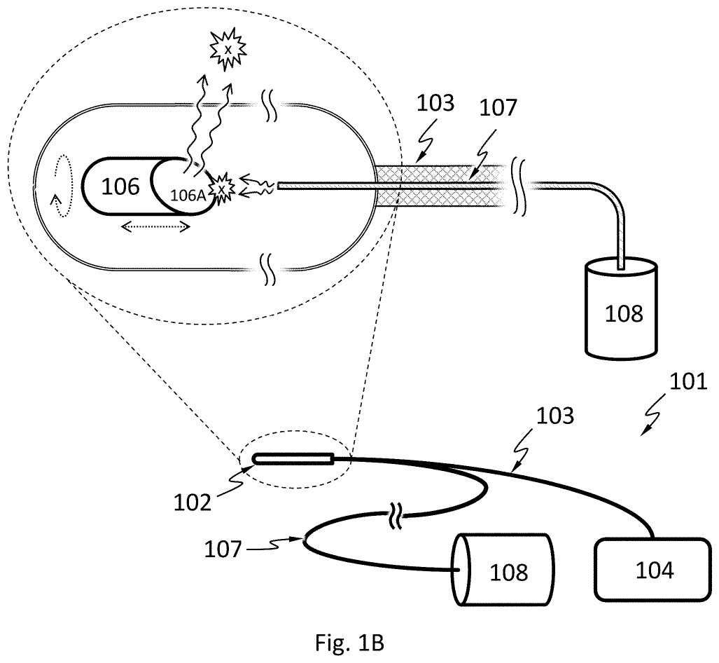

FIG. 1A and FIG. 1B each schematically show a functional diagram of an apparatus 101, according to an embodiment. The apparatus 101 has an insertion tube 102. The insertion tube 102 is configured to be inserted into a human. The word “inserted” can encompass “fully inserted” or “partially inserted.” The insertion tube 102 may have a small diameter (e.g., less than 50 mm), which makes it suitable for inserting into the rectum of the human.

The insertion tube 102 has a metal target 106 disposed therein. The metal target 106 may be hermetically sealed for protection from bodily fluid in the human. The metal target 106 can emit X-ray by receiving radiation. At least part of the insertion tube 102 is transparent to the X-ray. The insertion tube 102 may be opaque to the radiation. The metal target 106 may include tungsten, rhenium, molybdenum, copper, a combination thereof or other suitable metals. The metal target 106 may move along the insertion tube 102, or rotate relative to the insertion tube 102 (e.g., about an axis of the insertion tube 102). The metal target 106 may have an angled surface 106A with respect to the insertion tube 102. The angled surface 106A receives the radiation and emits the X-ray. The metal target 106 may be oriented (e.g., by moving it or rotating it) such that the X-ray it emits is directed toward a portion of the human. The portion of the human may be the prostate. The photons of the X-ray may have energies between 20 keV and 30 keV. According to an embodiment, a portion or the entirety of the insertion tube 102 may be inserted into the human. While the portion or the entirety of the insertion tube 102 is inserted into the human, the metal target 106 may be also inside the human. For example, the metal target 106 may be positioned at the distal end of the insertion tube 102.

As shown in FIG. 1A, the radiation is electrons and the insertion tube 102 has an electron emitter 105 inside, according to an embodiment. The electron emitter 105 may be based on any suitable mechanism (e.g., field emission or thermionic emission) for emitting electrons. The electron emitter 105 can accelerate the electrons toward the metal target 106, e.g., by an electric potential (e.g., 30 kV to 150 kV) between the electron emitter 105 and the metal target 106. A portion of the insertion tube 102 containing the electron emitter 105 may be configured to be left outside the human while the portion of the insertion tube 102 containing the metal target 106 is inserted into the human.

The apparatus 101 may have a signal cable 103 and a control unit 104. The control unit 104 may be configured to receive or transmit signals or control the movement of the insertion tube 102, through the signal cable 103. In the example in FIG. 1A, the control unit 104 may be configured to control the movement of the metal target 106 inside the insertion tube 102, to supply power to the electron emitter 105, or to establish the electric potential between the electron emitter 105 and the metal target 106, through the signal cable 103.

As shown in FIG. 1B, the radiation is an electromagnetic radiation such as another X-ray or gamma ray, according to an embodiment. The electromagnetic radiation may have higher photon energy (i.e., shorter wavelength) than the X-ray the metal target 106 emits. The apparatus 101 may have a radiation source 108 outside the insertion tube 102 and configured to generate the electromagnetic radiation. The radiation source 108 may remain outside of the human when the insertion tube 102 is inserted into the human. In an example, the apparatus 101 has polycapillary lenses 107 connecting the radiation source 108 and the insertion tube 102. The polycapillary lenses 107 are arrays of small hollow tubes (e.g., glass tubes) that guide certain electromagnetic radiation with many total reflections on the inside of the tubes. The electromagnetic radiation from the radiation source 108 may be directed toward the metal target 106 by the polycapillary lenses 107 or other suitable optics. The electromagnetic radiation, when received by the metal target 106, may cause the metal target 106 to emit the X-ray due to X-ray fluorescence. X-ray fluorescence (XRF) is the emission of characteristic fluorescent X-ray from a material that has been excited by, for example, exposure to high-energy X-rays or gamma rays. An electron on an inner orbital of an atom of the material may be ejected, leaving a vacancy on the inner orbital, if the atom is exposed to X-rays or gamma rays with photon energy greater than the ionization potential of the electron. When an electron on an outer orbital of the atom relaxes to fill the vacancy on the inner orbital, an X-ray (fluorescent X-ray or secondary X-ray) is emitted. The emitted X-ray has a photon energy equal the energy difference between the outer orbital and inner orbital electrons.

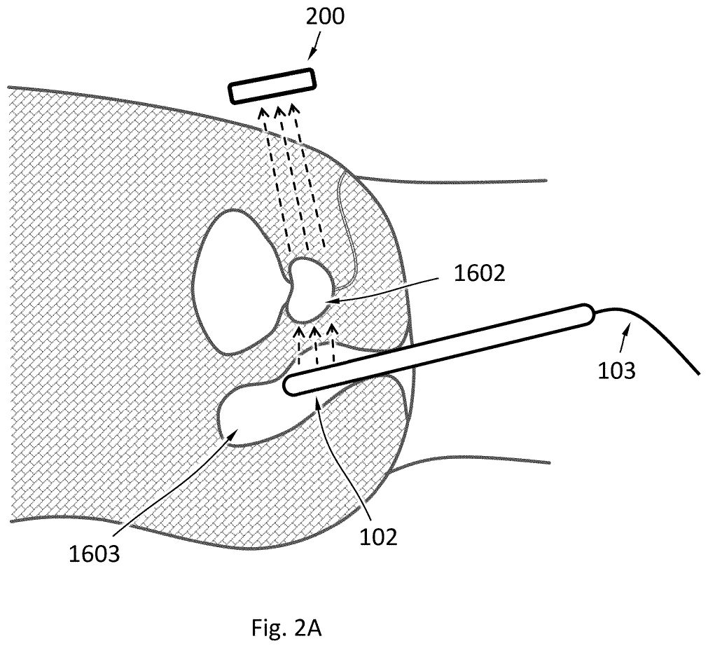

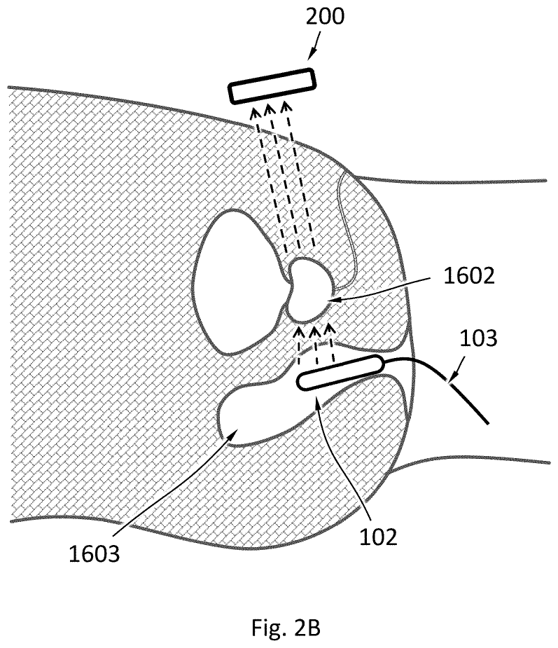

The apparatus 101 may further include an image sensor 200 configured to capture an image of the portion of the human (e.g., the prostate) using the X-ray emitted from the metal target 106 in the insertion tube 102, according to an embodiment, as shown in FIG. 2A and FIG. 2B.

FIG. 2A and FIG. 2B each schematically show a system including the apparatus 101 described above and an image sensor 200, according to an embodiment. The insertion tube 102 may be inserted partially or fully into the rectum 1603 of a human. The image sensor 200 may form an image of the prostate 1602 with X-ray emitted from the metal target 106 and transmitted through the prostate 1602. The system may be used for radiography on the prostate 1602.

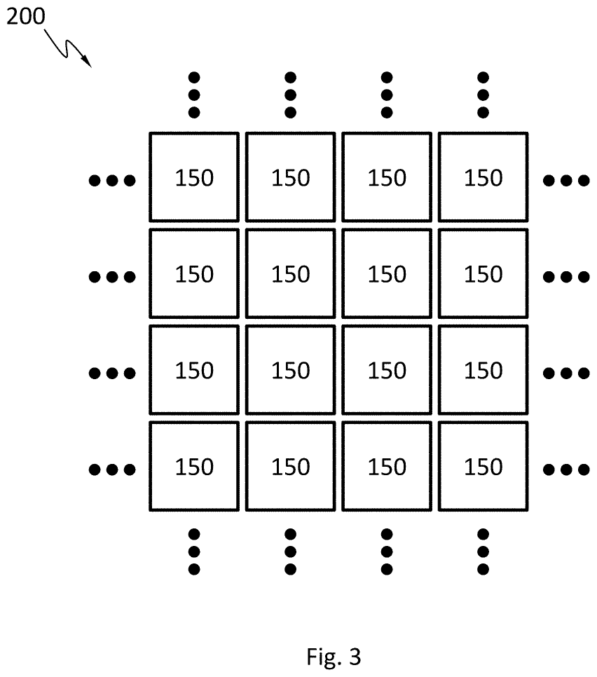

FIG. 3 schematically shows that the image sensor 200 may have an array of pixels 150, according to an embodiment. The array of the pixels 150 may be a rectangular array, a honeycomb array, a hexagonal array or any other suitable array. The image sensor 200 may count the numbers of photons of X-ray incident on the pixels 150, within a period of time. Each pixel 150 may be configured to measure its dark current, such as before or concurrently with each particle of radiation incident thereon. The pixels 150 may be configured to operate in parallel. For example, the image sensor 200 may count one photon of X-ray incident on one pixel 150 before, after or while the image sensor 200 counts another photon of X-ray incident on another pixel 150. The pixels 150 may be individually addressable.

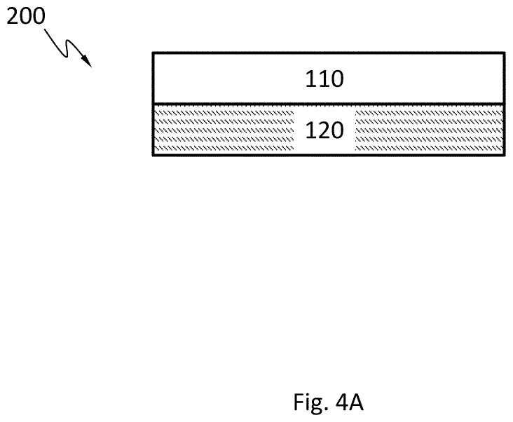

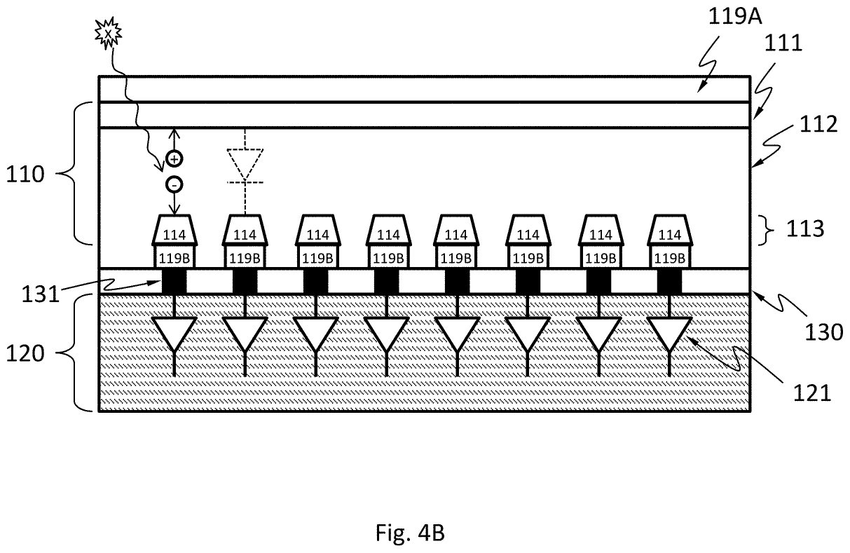

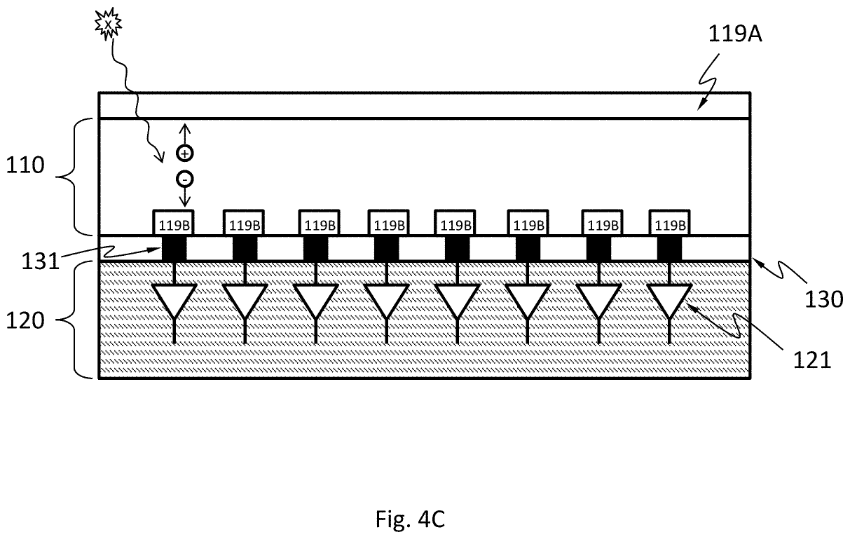

FIG. 4A shows a cross-sectional schematic of the image sensor 200, according to an embodiment. The image sensor 200 may include a radiation absorption layer 110 and an electronics layer 120 (e.g., an ASIC) for processing or analyzing electrical signals incident particles of radiation generate in the radiation absorption layer 110. The image sensor 200 may or may not include a scintillator. The radiation absorption layer 110 may include a semiconductor material such as single-crystalline silicon. The semiconductor may have a high mass attenuation coefficient for the radiation of interest.

As shown in a more detailed cross-sectional schematic of the image sensor 200 in FIG. 4B, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 4B, each of the discrete regions 114 of the second doped region 113 forms a diode with the first doped region 111 and the optional intrinsic region 112. Namely, in the example in FIG. 4B, the radiation absorption layer 110 has a plurality of diodes having the first doped region 111 as a shared electrode. The first doped region 111 may also have discrete portions. The radiation absorption layer 110 may have an electric contact 119A in electrical contact with the first doped region 111. The radiation absorption layer 110 may have multiple discrete electric contacts 119B, each of which is in electrical contact with the discrete regions 114.

When particles of radiation hit the radiation absorption layer 110 including diodes, the particles of radiation may be absorbed and generate one or more charge carriers by a number of mechanisms. The charge carriers may drift to the electric contacts 119A and 119B under an electric field. The field may be an external electric field. In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

As shown in an alternative detailed cross-sectional schematic of the image sensor 200 in FIG. 4C, according to an embodiment, the radiation absorption layer 110 may include a resistor of a semiconductor material such as single-crystalline silicon but does not include a diode. The semiconductor may have a high mass attenuation coefficient for the radiation of interest. The radiation absorption layer 110 may have an electric contact 119A in electrical contact with the semiconductor on one surface of the semiconductor. The radiation absorption layer 110 may have multiple electric contacts 119B on another surface of the semiconductor.

When particles of radiation hit the radiation absorption layer 110 including a resistor but not diodes, the particles of radiation may be absorbed and generate one or more charge carriers by a number of mechanisms. A particle of the radiation may generate 10 to 100000 charge carriers. The charge carriers may drift to the electrical contacts 119A and 119B under an electric field. The field may be an external electric field. In an embodiment, the charge carriers may drift in directions such that the charge carriers generated by a single particle of the radiation are not substantially shared by two electrical contacts 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of the electrical contacts 119B are not substantially shared with another of the electrical contacts 119B. A pixel 150 associated with one of the electrical contacts 119B may be an area around it in which substantially all (more than 98%, more than 99.5%, more than 99.9% or more than 99.99% of) charge carriers generated by a particle of the radiation incident therein flow to that one electrical contact 119B. Namely, less than 2%, less than 0.5%, less than 0.1%, or less than 0.01% of these charge carriers flow beyond the pixel associated with that one electrical contact 119B.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs. The electronic system 121 may include components shared by the pixels or components dedicated to a single pixel. For example, the electronic system 121 may include an amplifier dedicated to each pixel 150 and a microprocessor shared among all the pixels 150. The electronic system 121 may be electrically connected to the pixels by vias 131. Space among the vias may be filled with a filler material 130, which may increase the mechanical stability of the connection of the electronics layer 120 to the radiation absorption layer 110. Other bonding techniques are possible to connect the electronic system 121 to the pixels without using vias.

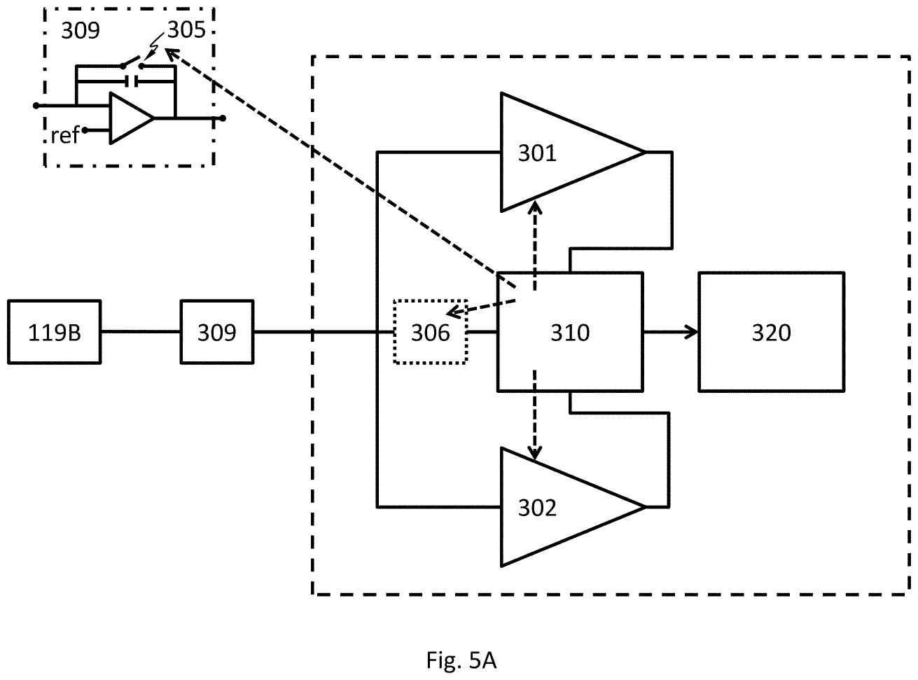

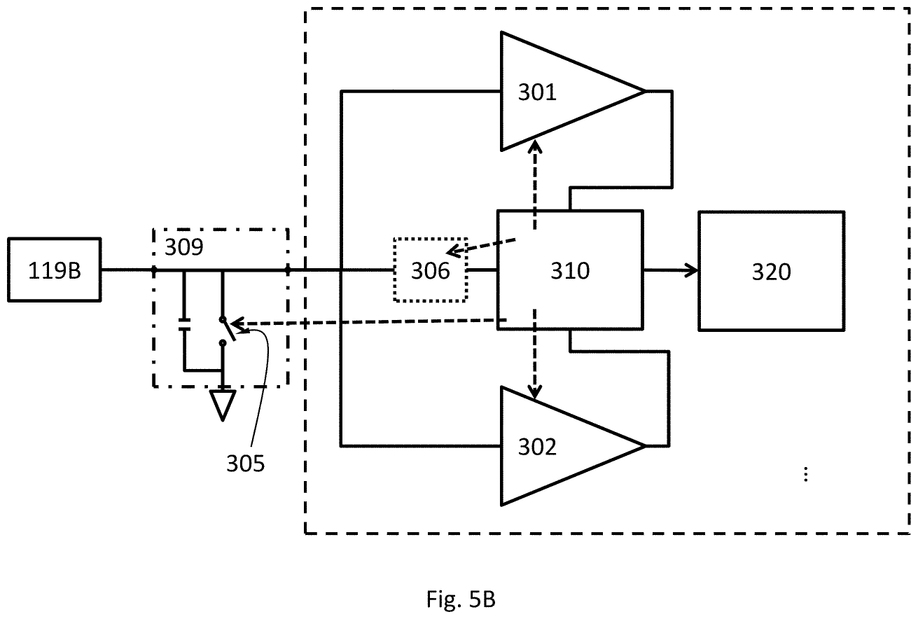

FIG. 5A and FIG. 5B each show a component diagram of the electronic system 121, according to an embodiment. The electronic system 121 may include a first voltage comparator 301, a second voltage comparator 302, a counter 320, a switch 305, an optional voltmeter 306 and a controller 310.

The first voltage comparator 301 is configured to compare the voltage of at least one of the electric contacts 119B to a first threshold. The first voltage comparator 301 may be configured to monitor the voltage directly, or calculate the voltage by integrating an electric current flowing through the electrical contact 119B over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 may be a clocked comparator. The first threshold may be 5-10%, 10%-20%, 20-30%, 30-40% or 40-50% of the maximum voltage one incident particle of radiation may generate on the electric contact 119B. The maximum voltage may depend on the energy of the incident particle of radiation, the material of the radiation absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electrical contact over a period of time. The second voltage comparator 302 may be a continuous comparator. The second voltage comparator 302 may be controllably activate or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” |x| of a real number x is the non-negative value of x without regard to its sign. Namely,

$$\vert x \vert = \{ \begin{matrix}x , if x \geq 0 \\ - x , if x \leq 0\end{matrix} .$$

The second threshold may be 200%-300% of the first threshold. The second threshold may be at least 50% of the maximum voltage one incident particle of radiation may generate on the electric contact 119B. For example, the second threshold may be 100 mV, 150 mV, 200 mV, 250 mV or 300 mV. The second voltage comparator 302 and the first voltage comparator 310 may be the same component. Namely, the system 121 may have one voltage comparator that can compare a voltage with two different thresholds at different times.

The first voltage comparator 301 or the second voltage comparator 302 may include one or more op-amps or any other suitable circuitry. The first voltage comparator 301 or the second voltage comparator 302 may have a high speed to allow the system 121 to operate under a high flux of incident particles of radiation. However, having a high speed is often at the cost of power consumption.

The counter 320 is configured to register at least a number of particles of radiation incident on the pixel 150 encompassing the electric contact 119B. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electrical contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In an embodiment, the controller 310 is configured to activate the second voltage comparator at the beginning of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause the number registered by the counter 320 to increase by one, if, during the time delay, the second voltage comparator 302 determines that the absolute value of the voltage equals or exceeds the absolute value of the second threshold.

The controller 310 may be configured to cause the optional voltmeter 306 to measure the voltage upon expiration of the time delay. The controller 310 may be configured to connect the electric contact 119B to an electrical ground, so as to reset the voltage and discharge any charge carriers accumulated on the electric contact 119B. In an embodiment, the electric contact 119B is connected to an electrical ground after the expiration of the time delay. In an embodiment, the electric contact 119B is connected to an electrical ground for a finite reset time period. The controller 310 may connect the electric contact 119B to the electrical ground by controlling the switch 305. The switch may be a transistor such as a field-effect transistor (FET).

In an embodiment, the system 121 has no analog filter network (e.g., a RC network). In an embodiment, the system 121 has no analog circuitry.

The voltmeter 306 may feed the voltage it measures to the controller 310 as an analog or digital signal.

The electronic system 121 may include an integrator 309 electrically connected to the electric contact 119B, wherein the integrator is configured to collect charge carriers from the electric contact 119B. The integrator 309 can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electric contact 119B accumulate on the capacitor over a period of time (“integration period”). After the integration period has expired, the capacitor voltage is sampled and then reset by a reset switch. The integrator 309 can include a capacitor directly connected to the electric contact 119B.

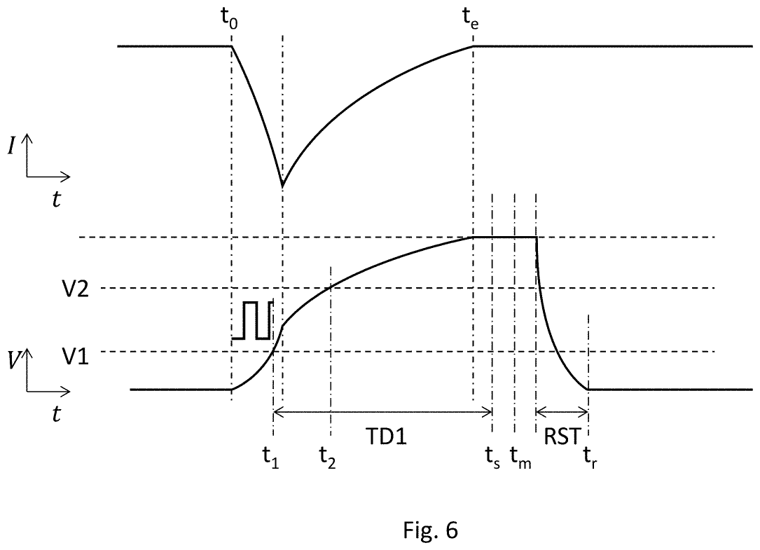

FIG. 6 schematically shows a temporal change of the electric current flowing through the electric contact 119B (upper curve) caused by charge carriers generated by a particle of radiation incident on the pixel 150 encompassing the electric contact 119B, and a corresponding temporal change of the voltage of the electric contact 119B (lower curve). The voltage may be an integral of the electric current with respect to time. At time t₀, the particle of radiation hits pixel 150, charge carriers start being generated in the pixel 150, electric current starts to flow through the electric contact 119B, and the absolute value of the voltage of the electric contact 119B starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 waits for stabilization of the voltage to stabilize. The voltage stabilizes at time tₑ, when all charge carriers generated by the particle of radiation drift out of the radiation absorption layer 110. At time tₛ, the time delay TD1 expires. At or after time tₑ, the controller 310 causes the voltmeter 306 to digitize the voltage and determines which bin the energy of the particle of radiation falls in. The controller 310 then causes the number registered by the counter 320 corresponding to the bin to increase by one. In the example of FIG. 6, time tₛ is after time tₑ; namely TD1 expires after all charge carriers generated by the particle of radiation drift out of the radiation absorption layer 110. If time tₑ cannot be easily measured, TD1 can be empirically chosen to allow sufficient time to collect essentially all charge carriers generated by a particle of radiation but not too long to risk have another incident particle of radiation. Namely, TD1 can be empirically chosen so that time tₛ is empirically after time tₑ. Time tₛ is not necessarily after time tₑ because the controller 310 may disregard TD1 once V2 is reached and wait for time tₑ. The rate of change of the difference between the voltage and the contribution to the voltage by the dark current is thus substantially zero at tₑ. The controller 310 may be configured to deactivate the second voltage comparator 302 at expiration of TD1 or at t₂, or any time in between.

The voltage at time tₑ is proportional to the amount of charge carriers generated by the particle of radiation, which relates to the energy of the particle of radiation. The controller 310 may be configured to determine the energy of the particle of radiation, using the voltmeter 306.

After TD1 expires or digitization by the voltmeter 306, whichever later, the controller 310 connects the electric contact 119B to an electric ground for a reset period RST to allow charge carriers accumulated on the electric contact 119B to flow to the ground and reset the voltage. After RST, the system 121 is ready to detect another incident particle of radiation. If the first voltage comparator 301 has been deactivated, the controller 310 can activate it at any time before RST expires. If the controller 310 has been deactivated, it may be activated before RST expires.

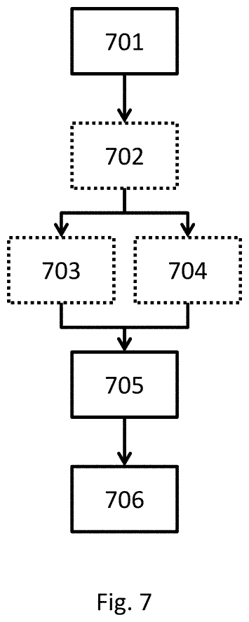

FIG. 7 shows a flowchart for a method using the apparatus 101, according to an embodiment.

In procedure 701, the insertion tube 102 with the metal target 106 is inserted into a human (e.g., into the rectum of the human). In optional procedure 702, the metal target 106 is moved along the insertion tube 102 or rotated with respect to the insertion tube 102. From here, the flow may go to optional procedure 703 when the radiation received by the metal target 106 is electrons, or go to optional procedure 704 when the radiation received by the metal target 106 is an electromagnetic radiation. In optional procedure 703, the electrons are produced outside the human (e.g., by the electron emitter 105). In optional procedure 704, the electromagnetic radiation is produced outside the human (e.g., by the radiation source 108). In procedure 705, X-ray is emitted from the metal target 106 by directing the radiation onto the metal target 106. In procedure 706, the portion (e.g., the prostate) of the human is imaged with the X-ray from the metal target 106.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An apparatus comprising:
an insertion tube configured to be inserted into a human;
a metal target disposed inside the insertion tube and configured to emit X-ray by receiving radiation.

2. The apparatus of claim 1, wherein the insertion tube is configured to be inserted into the rectum of the human.

3. The apparatus of claim 1, wherein the metal target is configured to be inside the human when the insertion tube is inserted into the human.

4. The apparatus of claim 1, wherein the metal target is configured to move along the insertion tube.

5. The apparatus of claim 1, wherein the metal target is configured to rotate relative to the insertion tube.

6. The apparatus of claim 1, wherein the metal target comprises an angled surface configured to receive the radiation.

7. The apparatus of claim 1, wherein the radiation is electrons.

8. The apparatus of claim 7, further comprising an electron emitter disposed inside the insertion tube, configured to emit the electrons, and configured to accelerate the electrons toward the metal target.

9. The apparatus of claim 8, wherein the electron emitter is configured to be left outside of the human when the insertion tube is inserted into the human.

10. The apparatus of claim 1, wherein the radiation is an electromagnetic radiation.

11. The apparatus of claim 10, wherein the electromagnetic radiation is another X-ray.

12. The apparatus of claim 10, wherein the metal target is configured to emit X-ray due to fluorescence caused by the electromagnetic radiation.

13. The apparatus of claim 10, further comprising polycapillary lenses configured to direct the electromagnetic radiation toward the metal target.

14. The apparatus of claim 10, further comprising a radiation source configured to produce the electromagnetic radiation.

15. The apparatus of claim 14, wherein the radiation source is configured to be left outside of the human when the insertion tube is inserted into the human.

16. The apparatus of claim 1, wherein photons of the X-ray have energies between 20 keV and 30 keV.

17. The apparatus of claim 1, further comprising an image sensor configured to capture an image of a portion of the human using the X-ray.

18. A method comprising:
inserting an insertion tube with a metal target therein into a human;
emitting X-ray from the metal target by directing radiation onto the metal target;
imaging a portion of the human with the X-ray.

19. The method of claim 18, wherein inserting the insertion tube into the human comprises inserting the insertion tube into the rectum of the human.

20. The method of claim 18, wherein the portion is the prostate.

21. The method of claim 18, further comprising moving the metal target along the insertion tube or rotating the metal target with respect to the insertion tube.

22. The method of claim 18, wherein photons of the X-ray have energies between 20 keV and 30 keV.

23. The method of claim 18, wherein the radiation is electrons.

24. The method of claim 23, further comprising producing the electrons outside the human.

25. The method of claim 18, wherein the radiation is an electromagnetic radiation.

26. The method of claim 25, wherein the electromagnetic radiation is another X-ray.

27. The method of claim 25, wherein emitting X-ray from the metal target is by fluorescence of the metal target caused by the electromagnetic radiation.

28. The method of claim 25, further comprising producing the electromagnetic radiation outside the human.

29. The method of claim 25, wherein directing the radiation onto the metal target is by using polycapillary lenses.
